(19) United States
(12) Patent Application Publication
(10) Pub. No.: US 2008/0254067 A1
(43) Pub. Date: Oct. 16, 2008

(54) Process for the Production of an Influenza Vaccine

(75) Inventors: Pierre Trepanier, Laval (CA); Robert Dugre, Ste-Therese (CA); Tom Hassell, North York (CA)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: ID BIOMEDICAL CORPORATION, QUEBEC CANADA (CA)

(21) Appl. No.: 11/596,835

(22) PCT Filed: May 20, 2005
(86) PCT No.: PCT/US05/17606

§ 371 Date: Mar. 30, 2008

Related U.S. Application Data
(60) Provisional application No. 60/572,612, filed on May 20, 2004.

(51) Int. Cl. A61K 39/145; C12N 5/06; C12N 7/02

(52) U.S. Cl. 424/209.1; 435/350; 435/325; 435/239

(57) ABSTRACT

The present invention relates to a commercial-scale process for the production of influenza virus or antigens for prophylactic, diagnostic, immunotherapeutic or therapeutic purposes. Particularly, the invention provides a Madin-Darby Canine Kidney (MDCK)-derived, cell line and a cell culture-based process for the production of an influenza vaccine and more particularly, a human vaccine comprising influenza types A and B.

This application claims the benefit of U.S. provisional application 60/572,612 filed May 20, 2004, which is herein incorporated by reference.

### FIELD OF THE INVENTION

The present invention generally relates to a commercial-scale process for the production of influenza virus or antigens for prophylactic, diagnostic, immunotherapeutic or therapeutic purposes. Particularly, the invention provides a Madin-Darby Canine Kidney (MDCK)-derived, cell culture-based process for the production of an influenza vaccine and more particularly, a human vaccine comprising influenza types A and B.

### BACKGROUND OF THE INVENTION

Traditionally, commercial influenza vaccines have been produced by growing vaccine virus strains in embryonated hens' eggs. The virus is harvested from the allantoic fluid and processed to create a vaccine. However, this procedure has the disadvantages of being labour-intensive and generating low yields per egg, factors which present a serious limitation during periods of epidemic. There is thus a need for the large-scale manufacture of influenza virus vaccines which overcomes the cost, time and yield disadvantages of the embryonated hen egg method.

An alternative method to the above involves the use of cell culture to produce influenza virus particles or virus proteins.

Influenza vaccines produced with cell cultures are believed to be safer than ones produced in eggs and should not induce the hypersensitivity to egg-based vaccines experienced by children and adults. Such vaccines should also confer a better protection to a wider spectrum of wild strains, particularly in the elderly, because fidelity of viral replication is thought to be greater in cell cultures than in eggs (Katz, J. M., et al, J. Infect Diseases 160:191, 1989). Moreover, in cases of epidemic or pandemic, a greater supply of influenza vaccine can be produced than is currently possible due to limitations in egg supply.

The propagation of influenza A and B viruses has been demonstrated in a variety of tissue-culture systems including minced chick embryo, human embryo lung and kidney, monkey kidney and bovine embryo kidney.

In particular, canine kidney cells have been suggested as useful for the production of influenza virus, albeit at low yields, i.e. insufficient for vaccine production purposes.

Canine kidney cells were originally derived in 1958 by S. H. Madin and N. B. Darby from a kidney of an apparently normal adult female Cocker Spaniel (American Type Culture Collection (ATCC), Catalogue of Cell Lines and Hybridomas, 7th edition, p. 21, 1992). The MDCK cell line was deposited with the ATCC in 1964 (under No. CCL 34) and identified as allowing the replication of several viruses, including vesicular stomatitis, vaccinia, Coxsackie, reovirus, and adenovirus.

The serial propagation of influenza virus type B was first demonstrated in MDCK cell line by Green in 1962 (Science 138:42, 1962). Propagation was evidenced by the presence of cytopathic effects and hemagglutinin (HA), the major glycoprotein of influenza virus, in each of six consecutive tissue-culture passages, and by egg-infectivity titers in tissue culture fluids. However, these data are limited to small scale and do not provide a means of achieving large-scale production of viral particles or proteins for vaccine purposes. Gaush et al. in 1966 demonstrated that MDCK cells were also susceptible to influenza A infection. However, this paper reported infectivity only and did not address the issue of propagation of the virus in this medium (Gaush et al., Proc. Soc. Exp. Biol. and Med., 122: 931, 1966).

In 1975, Tobita et al. described for the first time the growth of a wide variety of influenza A viruses in an established MDCK cell line in an overlay medium containing trypsin. The virus propagation formed well defined plaques regardless of their prior passage history and it was proposed that trypsin contributed to the cleavage of HA polypeptide thereby accelerating the maturation of influenza virus. Yet despite the advancement that the use of trypsin provided, the isolation of virus in agar medium did not provide a means of attaining a large-scale production of the virus. In the same reference, MDCK cells were also used successfully for the primary isolation of influenza A virus from throat washings of patients.

Subsequently, Reuveny et al. grew influenza virus type A on MDCK cells on cellulose-based microcarriers in batch culture (Develop. Biol. Standard 50:115, 1982). The influenza virus titer obtained was similar in trypsin-containing medium as in embryonated eggs.

U.S. Pat. No. 4,500,513 (Brown et al.) describes a method for the replication of influenza virus in successive numbers of cells of the same liquid culture by including a protein hydrolyzing enzyme such as trypsin in the culture during virus incubation. The proteolytic enzyme is required to render HA functional and thereby overcome the one-step growth cycle of past liquid culture techniques. This is the first description of a potential “commercial” influenza vaccine production from liquid cell culture.

However, it is now considered that the presence of trypsin in solution has the disadvantage of causing a certain proportion of MDCK cells to lift from their solid support. Consequently, the requirement for trypsin is a serious limitation to the commercial production of an influenza vaccine, despite the potential usefulness of this patented process. There is still a need therefore for a commercial cell culture-based process for the production of an influenza vaccine. This is supported by the following statement in Kodihalli et al. (J. Virol. 69(8): 4888, 1995): “Embryonated chicken eggs are currently the only host in which sufficient quantities of virus can be cultivated economically and within the short time necessary to ensure a vaccine supply”.

Also in 1995, the World Health Organization (WHO) published a memorandum summarizing discussions that took place in the summer of 1995, when experts in the field met to evaluate the recent advancements relating to cell culture-based processes for the production of influenza vaccines (Bull. W.H.O. 73(4): 431, 1995). This document makes recommendations for further work to achieve rapid production of large amounts of influenza vaccine in cell culture systems which would allow rapid scale-up.

For growth of viruses in cell culture for research purposes, MDCK and Vero cells are the most frequently referred to as good producers of several viruses. However, for large scale production purposes several factors influence the choice of a particular cell line over another, such as susceptibility to one or a multiplicity of viruses, output of viral titers, anchorage-dependency, tumorigenicity, etc.

Although MDCK cells are susceptible to several viral strains, the poor resulting titers may limit the usefulness of this cell line for large-scale production purposes.

The properties of the MDCK line have been the subject of some studies. In 1970, Leighton et al (Cancer 26:1022) reported that MDCK cells presented the morphological pattern of a papillary adenocarcinoma in histopathologic preparations of three dimensional tissue cultures on collagen-coated cellulose sponge. The neoplastic quality of the cell line was demonstrated when suspensions of cells which had been injected in 11- or 12-day-old chick embryos were found to produce many foci of brain metastasis.

Evaluation of the tumorigenicity of a cell line is important in order to assess its desirability for use in the production of biological products. The Center for Biologics Evaluation and Research of the US Food and Drug Administration has published points to consider when using a cell culture as a substrate for the production of biologicals (See: Points to Consider in the Characterization of Cell Lines Used to Produce Biologicals, Office of Biologics Research and Review, Center for Drugs and Biologics, FDA (USA), 1993). One such point is the tumorigenicity of the cell line used, and the FDA has devised guidelines for in vivo tumorigenicity testing. These guidelines call for, among other things, testing of cells administered by subcutaneous or intramuscular route in nude mice (nu/nu).

### SUMMARY OF THE INVENTION

Accordingly, the present invention relates to a process for the large-scale production of influenza viral particles or proteins, comprising the steps of:
(a) growing a MDCK cell line capable of replicating said influenza virus;
(b) infecting the MDCK cell culture with a strain of influenza virus and incubating to allow replication of the virus;
(c) harvesting said replicated virus and purifying virus particles or proteins therefrom.

The invention further provides for a cell line derived from the MDCK cell line that is highly-susceptible to viral infection and which produces influenza virus in higher titer than its parental cell line.

### BRIEF DESCRIPTION OF THE FIGURES

FIG. 1 is a front sectional view of a decanter used in the perfusing means of the invention;

FIG. 2 is a top elevational view of said decanter; and

FIG. 3 is a bottom elevational view of said decanter.

### DETAILED DESCRIPTION OF THE INVENTION

In one embodiment, the MDCK-derived cell line of the present invention, allows multi-step replication of the influenza virus. In another embodiment, the MDCK cell line of the present invention is anchorage-dependent and non-tumorigenic.

The present invention provides a derivation of a MDCK clone that is super-susceptible to viral infection. The description “super-susceptible” is used to indicate a MDCK-derived cell line that is highly susceptible to at least one virus, thereby producing higher titers of viral particles than the parental MDCK cell line. This derived clone, MDCK.5F1, was deposited with the American Type Culture Collection (ATCC) on Feb. 8, 1996 under No. CRL-12042. In one embodiment, the MDCK-derived cell line of the present invention is non-tumorigenic in tests conducted in accordance with FDA guidelines, and thus may be suitable for use in the preparation of viruses or antigens for prophylactic, diagnostic, immunotherapeutic or therapeutic purposes. The cell line has been tested for the presence of contaminating microorganisms and none have been detected.

In accordance with the present invention there is also provided, a process for the large-scale production of influenza virus particles or proteins for the manufacture of a vaccine.

In accordance with an aspect of the present invention there is also provided, a large-scale, cell-cultured, microcarrier-based commercial process for the production of influenza virus particles or proteins for the manufacture of influenza vaccines.

The process of this invention is carried out using a MDCK cell line for the multiple replication of the influenza virus. In one embodiment, the MDCK cell line used in the process of this invention has the same biological properties as those of ATCC cell line No. CRL-12042. In one embodiment, the cell line used in the process is a clone of the MDCK cell line internally designated as MDCK.5F1, deposited under ATCC No. CRL-12042.

The MDCK-derived cell line of this invention is highly susceptible to viral infection. “Highly susceptible” to viral infection in this context means that the cell line is capable of producing titers that are higher than the titers produced by the parental cell line for at least one viral strain.

In one embodiment, “high susceptibility” is defined as a cell line capable of producing a virus at a titer of at least about 1.2 times the titer produced by the parental cell line. In one embodiment, the higher susceptible cell lines are clones selected from the group consisting of: 3B5, 5F1, 1D11, 5H12, 9C2, 9D9, P79, 9E9, 7C1, and P123.

Still, in one embodiment, higher susceptibility is defined as a cell line capable of producing a multiplicity of viruses at a titer of at least about twice the titer produced by the parental cell line for these same viruses. In one embodiment, such a clone is selected from the group consisting of: 3B5, 5F1, 5H12, 9C2, 7C1, and P123.

In one embodiment, higher susceptibility is defined as capable of producing at least about twice the parental viral titer for two different strains of the same virus. In one embodiment, such a clone is capable of producing twice the viral titer of the parent for respiratory syncytial virus and influenza types A and B. In one embodiment, such a clone is selected from the group consisting of 5F1 and 5H12.

In one embodiment, the cell line is capable of being infected by a virus selected from the group consisting of: influenza, respiratory syncytial virus, papovavirus, parainfluenza, vesicular stomatitis, vaccinia, Coxsackie, reovirus, parvovirus, adenovirus, poliomyelitis, measles, rabies, herpes, and other viruses.

In one embodiment, the virus is selected from the group consisting of: influenza types A, B, and C; respiratory syncytial virus; papovavirus; vesicular stomatitis (Indiana strain); Coxsackie B-5; reovirus types 2, and 3; and adenovirus types 4, and 5.

In one embodiment, the virus is selected from the group consisting of: influenza types A, B, and C, and respiratory syncytial virus.

In one embodiment, the virus is selected from human, equine, porcine or avian influenza strains.

In one embodiment, the virus is selected from human influenza types A, B or C.

In one embodiment, the cell line of the invention is capable of being infected by human influenza virus type A or B.

In one embodiment, the cell line of the invention is capable of being infected by both human influenza virus types A and B.

In one embodiment, the cell line of the present invention allows multi-step replication of influenza virus with addition of a proteolytic enzyme such as trypsin, chymotrypsin, pepsin, pancreatin, papain, pronase and carboxypeptidase. In one embodiment, such a cell line allows multi-step replication of influenza virus with the addition of trypsin.

Alternatively, the cell line of the present invention allows multi-step replication of influenza virus without requiring the addition of a proteolytic enzyme such as trypsin, chymotrypsin, pepsin, pancreatin, papain, pronase and carboxypeptidase. In one embodiment, such a cell line allows multi-step replication of influenza virus without requiring the addition of trypsin.

It will be apparent to a person skilled in the art which viruses or virus strains may require the use of a proteolytic enzyme.

Although it is possible to culture the cell line of this invention in suspension, it is preferable that it grow in an anchorage-dependent manner. In one embodiment, it is also capable of growing on microcarrier beads, thereby allowing high concentrations of cells to be obtained in cell culture.

In one embodiment, the MDCK-derived cell line of this invention is non-tumorigenic. In one embodiment the cell line of the invention grows with minimal efficiency (i.e., <1% efficiency) in soft agar. In one embodiment, the cell line of the invention does not produce nodules in nude mice when observed for at least 3 months.

The present invention further contemplates the use of the cell line according to the invention for the production of viral particles or viral proteins in bulk quantity.

Such viral particles or proteins can be used in the manufacture of vaccines for the prevention of viral infections in a host. In one embodiment, said host is a mammal. Mammals include, for example, humans, equine, porcine species. In another embodiment, said host is a human. In a further embodiment, the host is an avian species (e.g., duck or chicken).

The process of this invention may be carried out in the presence or absence of trypsin, as long as the presence of trypsin does not affect the ability of anchorage-dependent MDCK cells to grow in culture. In one embodiment, the process is performed in the presence of trypsin at a concentration of about or lower than 4 μg/ml. Alternatively, the process is performed in the absence of trypsin.

The process described herein is intended for the production of respiratory syncytial virus and a variety of influenza viruses such as human, equine, porcine and avian strains of the influenza virus. In one embodiment, the process described herein is intended for the production of human influenza type A, B or C. In one embodiment, the process described herein is intended for the production of human influenza type A or B. In one embodiment, the process described herein is intended for the production of human influenza type A and type B by using an MDCK cell line that may be infected by either type of virus.

The process of this invention provides yields that are in the range of 4 μg HA/10⁶ MDCK cells. As is described in Example 9, this process yields virus protein amounts greater than 4 μg HA/10⁶ MDCK cells, more particularly in the range of 9 μg HA/10⁶ MDCK cells.

“Large-scale process” means a process for producing large amounts of influenza virus. Such a process is usually done in a bioreactor as opposed to a culture flask. Such bioreactor may be of varying size depending on the final yield/doses required. For example, such bioreactor may be of approximately 5 liters in size (with a working volume of about 4 liters), or it may be up to 5000 liters. Of course, as will be apparent for a person skilled in the art of large-scale cell culture-based vaccine production, all reactants, media, nutrients, cell concentrations, microcarrier concentrations, perfusion rates, etc., will be adjusted according to the size of the bioreactor.

The process of this invention may be executed in suspension culture but it is preferable that it be carried out on microcarrier means to increase the cell concentration (and thereby increase viral output). For example, microcarrier beads of the type usually known in the art as dextran polymers (Cytodex™) may be selected. These microcarriers may be used at concentrations ranging from about 5-25 g/L. In one embodiment, the microcarrier concentration is in the range of about 10-25 g/L. In one embodiment, the microcarrier concentration is in the range of about 15-20 g/L.

A perfusing means is introduced to maximize both cell growth and viral replication in the process. Perfusion allows for the constant supply of nutrients while simultaneously providing a means of avoiding the accumulation of potentially toxic by-products in the culture medium. Through perfusion, nutrient type and quantity may be varied during the various stages of the process. For instance, serum may be introduced to the cells during the growth phase but ideally should be eliminated at cell confluency and before introducing the virus. The perfusion flow rate is gradually increased during cell growth to provide adequate nutritional supplies. Perfusion is continued during viral replication.

The perfusion rate is adjusted to between about 0.5 to 4 bioreactor volumes/day, depending on the stage of the process.

The perfusing means is added to the bioreactor. It includes inlet means to continuously introduce a culture medium in the bioreactor and two outlet means to continuously remove spent culture medium from the bioreactor (thereby producing a continuous flow of the culture medium through the microcarrier suspension in the bioreactor), and a decanter associated with the outlet means. The decanter herein described and used in the process of the invention was specially designed for a 5 L (3.7 L working volume) bioreactor when it was realized that, with a perfusing means as proposed above and using a concentration of microcarriers in excess of 10 g/L, cell growth was limited by the tendency of the microcarriers to move upwardly and either escape through or clog the outlet means. Therefore, in order to achieve higher microcarrier concentrations, a decanter having the following features was built which minimizes formation of turbulence within the decanter and achieves a speed of microcarrier sedimentation which is faster than the upward flow speed of the suspension.

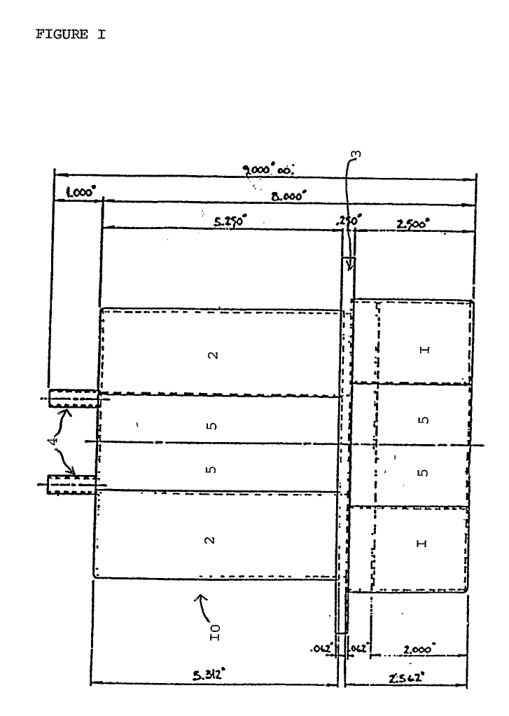

In accordance with a preferred embodiment of the invention, the decanter 10 as shown in FIG. 1, comprises a lower chamber 1 joined to a larger upper chamber 2 by a radial plate 3. The two outlet means 4 are attached to the upper chamber 2 at the top of the decanter. The lower chamber 1 and the upper chamber 2 are semi-cylindrical in shape and surround an axially directed central circular cavity 5 intended to accommodate the central rotating shaft of the bioreactor as well as various probes introduced in the bioreactor to monitor the process of the invention. Solid metal walls complete the decanter.

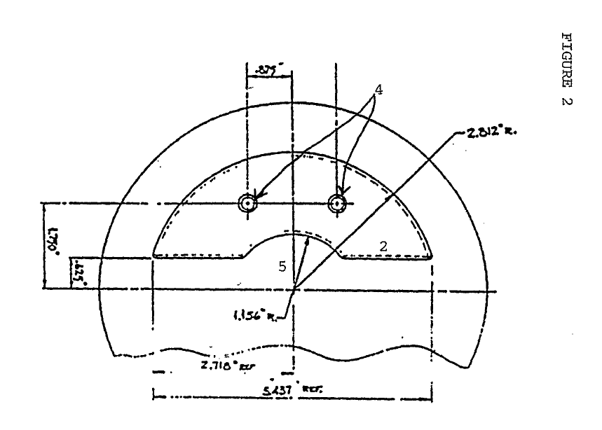

FIG. 2 illustrates a top elevational view of elements 2,4 and 5 described above.

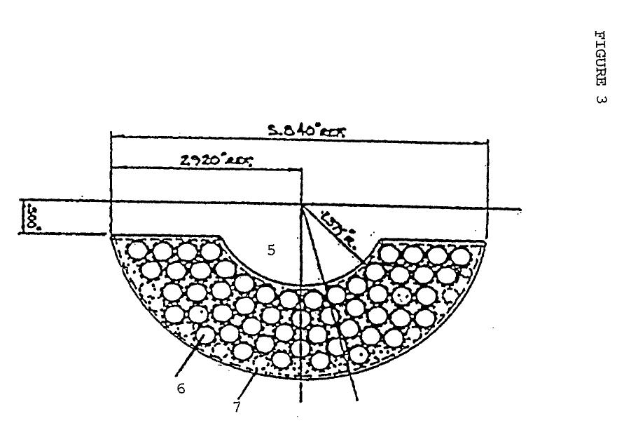

FIG. 3 shows a bottom elevational view of the lower chamber 1 of the decanter. The lower chamber 1 has a plurality of regularly distributed longitudinal channels 6 which are separated by radially oriented longitudinal partitions 7, the longitudinal channels being in communication with one another via the upper chamber 2 and the radial plate 3 of the decanter. In one embodiment the longitudinal channels 6 are identical and each has a circular cross-section. In addition, the plurality of longitudinal channels 6 are regularly distributed around the axially directed central circular cavity 5, with the surrounding longitudinal channels 6 being separated from one another by the radially oriented longitudinal partitions 7.

The outlet means of the perfusing means is switched from a waste reservoir to a harvest recipient at the moment of infection. Continuous harvesting occurs until replication is complete (about 4-5 days).

The process of the invention is carried out by seeding the MDCK cell culture with virus at a multiplicity of infection (M.O.I.) of between 10:1-1:10¹⁰. In one embodiment, the process is carried out at a M.O.I. of between 1:10-1:10⁸. In one embodiment, the process is carried out at a M.O.I of about 1:10⁴-1:10⁷. In one embodiment, the process is carried out at a M.O.I. of about 1:10⁵-1:10⁶.

In one embodiment, growth of MDCK cells in the process of this invention is carried out for about 7 to 10 days at a temperature of about 33-40° C. In one embodiment, growth of MDCK cells in the process is carried out for about 7 days at a temperature of about 36-38° C.

In one embodiment, growth of MDCK cells in the process is carried out for about 7 days at a temperature of about 37° C.

In one embodiment, viral replication in the process of this invention is carried out for about 4 to 6 days at a temperature of about 30-37° C. In one embodiment, viral replication in the process is carried out for about 5 days at a temperature of about 32-34° C. In one embodiment, viral replication in the process is carried out at a temperature of about 33° C.

Viral particles or proteins are purified in the following way. Filtration of the virus harvest is followed by inactivation of the filtrate with formaldehyde. The resulting inactivated viral suspension is then centrifuged and enriched virus fractions are selected for use in vaccine preparation.

A method for the prevention of influenza infection in a mammal, comprising the step of administering the vaccine of the invention is also provided.

A process wherein purified viral particles or proteins are used to produce a diagnostic kit for the detection of influenza virus infections is also provided.

The use of bulk virus particles or proteins isolated for the manufacture of a kit for the detection and diagnosis of influenza virus infection in a mammal is also provided.

Specific examples of the use of our process and propagation of selected strains of human influenza viruses types A and B are discussed below. It will be apparent that various changes may be made in the arrangement and components of the invention without departing from its essence and scope or sacrificing all of its material advantages, the forms hereinafter described being merely preferred or exemplary embodiments.

### EXAMPLES

Example 1 describes the derivation of clone MDCK.5F1. Example 2 considers the purity of the MDCK.5F1 cell line. Example 3 illustrates growth of influenza virus with and without trypsin on the MDCK.5F1 cell line. Example 4 is a summary of tumorigenicity studies on MDCK.5F1. Example 5 discusses results following the inoculation of nude athymic mice with a cell suspension of MDCK.5F1. Example 6 is a step-by-step description of a particular embodiment of the process. Example 7 describes the results of an assay performed using parental MDCK cells and influenza strain A/Shanghai/11/87 with trypsin. Example 8 describes the results of an assay performed using MDCK.5F1 cells and influenza strain A/Shanghai/11/87 without trypsin. Example 9 describes the results of an assay performed using MDCK.5F1 cells and influenza strain B/Harbin/7/94 without trypsin.

### Example 1

### Derivation of Clone MDCK.5F1

MDCK cells No. CCL 34 were obtained from the American Type Culture Collection, Rockville, Md. The stock was received in frozen state in 1 ml ampoules containing 3.4×10⁶ cells. The cell line was at its 54th passage.

After passaging, MDCK cells were harvested at passage 64 and diluted in a nutritive medium composed of Dulbecco's Modified Eagle Medium (DMEM) and Medium 199 in a 1:1 ratio (DMEM-199) containing 10% (v/v) fetal bovine serum (FBS). The diluted cell suspension was then aliquoted into 96-well plates, such that each well received less than one cell, assuming a uniform distribution of the cells in solution. The plates were placed in a CO₂ incubator at 37° C. and examined at weekly intervals under the light microscope in order to score the wells for growth.

The characteristics sought in the clone were selected from:
(1) higher susceptibility than parental line to viral infection (i.e., clone produces higher titers of virus than the parental line);
(2) higher susceptibility to more than one virus (In one embodiment, susceptibility to several strains of the influenza virus);
(3) the ability to allow multi-step replication of influenza virus without requiring the addition of a proteolytic enzyme such as trypsin; and, optionally,
(4) anchorage-dependency (i.e. to obtain higher concentrations of cells in culture).

Table 1 illustrates the susceptibility of several clones to infection by influenza type A and B viruses without the addition of trypsin.

TABLE 1
Clonal Susceptibility to Infection by Influenza Type
A and B Viruses

| | | Susceptibility |
|---|---|---|
| | Susceptibility to | to Type B |
| Clone Number* | Type A (TCID₅₀) | (TCID₅₀) |
| 3B5 | ND | 5.6× |
| 5F1 | 5.5× | 3.6× |
| 1D11 | 0.5× | 2.4× |
| 5H12 | 8.5× | 2.2× |
| 9C2 | 0.5× | 2.0× |
| 9D9 | ND | 1.5× |
| P79 | 1.0× | 1.0× |
| 8H7 | ND | 0.6× |
| 9E9 | 0 | 1.2× |
| 7C1 | 0.3× | 2.0× |
| P123 | ND | 2.8× |
| Control** | 1× | 1× |
| (parental MDCK) | | |

ND = Not Detected
Control = Parental susceptibility, defined as 1× when assessed by TCID₅₀
*= Clonal susceptibility was determined without the addition of trypsin in the culture medium
**= Control susceptibility was determined with the addition of trypsin

“High susceptibility” was defined as at least about 1.2 times the susceptibility of the parental cell line when assessed by TCID₅₀. Clones 3B5, 5F1, 1D11, 5H12, 9C2, 9D9, P79, 9E9, 7C1, and P123 were identified as being highly susceptible. Clones 3B5, 5F1, 1D11, 5H12, 9C2, 9D9, 7C1, and P123 were identified as being at least twice as susceptible as the parent line.

Clones 5F1 and 5H12 were selected as being the two most highly susceptible, and 5F1 was chosen to establish a cell line internally designated as MDCK.5F1.

Cell generation number was defined as zero at the point of cloning and calculated by cell enumeration at each subsequent cell culture. The culture was passaged initially in multi-well plate culture and eventually transferred to plastic flasks.

In addition, experiments were conducted with respiratory syncytial virus to measure the susceptibility to infection by this virus of the parental MDCK cell line and the MDCK.5F1 clone. Viral titers indicated that while the MDCK cell lines are much less susceptible to infection than the respiratory syncytial virus host Hep 2 cell line, the MDCK.5F1 clone was approximately ten times more susceptible to infection by this virus than was the parental MDCK cell line.

### Example 2

### Determination of Clonality of MDCK.5F1

The parameter which determines the clonality of any particular clone picked is the percentage growth on a multi-well plate from which the clone is selected. The probability of the culture selected actually being clonal (i.e., P(1)) is determined from this percentage. (See Coller and Coller, Methods in Enzymology, vol. 121, pp. 412-417 (Academic Press, 1986).)

Given that 5% of the wells showed growth in this cloning, the probability that cell line MDCK.5F1 is derived from a single cell is ≧97.5%.

A 299 ampoule Master Cell Bank (MCB) and a 283 ampoule Manufacturer's Working Cell Bank (WCB) were prepared from the MDCK.5F1 cell line. These banks were prepared in accordance with Canadian guidelines on the principles of Good Manufacturing Practice and were assessed for contamination in the form of fungal, yeast, mycoplasmal, bacterial and viral agents. No contamination of any kind was found.

Sustainable, viable cultures were obtained from the prepared cell banks and the cell line tested for stability of product production, morphology, tumorigencity, and isoenzyme characteristics for 50 population doublings beyond the WCB. The results showed the cell line to be stable for these characteristics.

### Example 3

### Experiments Illustrating Growth of Influenza Virus in MDCK.5F1 Clone with and without Addition of Trypsin

Experiments were performed to determine the ability of influenza viruses to reproduce in cultures of clone MDCK.5 μl in the presence and absence of trypsin. The results of three experiments using influenza strains A/Johannesburg, A/Texas and B/Harbin appear in Table 2.

TABLE 2
Growth of different influenza strains in MDCK.5F1
clone culture with and without trypsin

| | A/Johannesburg | | A/Texas | | B/Harbin | |
|---|---|---|---|---|---|---|
| Days | *HA | | *HA | | *HA | |
| post- | + | − | + | − | + | − |
| infection | trypsin | trypsin | trypsin | trypsin | trypsin | trypsin |
| 3 | 32 | 24 | 24 | 12 | 128 | 128 |
| 4 | 48 | 48 | 96 | 48 | 192 | 128 |
| 5 | 64 | 64 | 96 | 96 | 128 | 96 |

*Hemagglutination (HA) = a mean of two readings for 0.5% chick red blood cells expressed as the inverse of the final dilution after red blood cell addition

These results reveal that there is no substantial difference when influenza was made to replicate in cell cultures with and without trypsin, as shown by the hemagglutination (HA) titer values.

### Example 4

### Summary of In Vitro Tumorigenicity Studies

Following the method described by Furesz et al (Develop. Biol. Stand. vol. 70, pp. 233-243, S. Kargel ed., Basel, 1989), four ml of 0.6% agar in DMEM-199 medium with 10% (v/v) FBS was allowed to set in tissue culture dishes containing six wells of 35 mm diameter. After setting, these wells were overlaid with 3 ml of medium containing ungelled agar at 0.3% W/V maintained at 42° C. and a cell concentration of 60,000 cells/ml. Plates were incubated at 37° C. with 5% CO₂. Light microscope observations were made at days 3, 7, 10 and 14. Colonies consisted of four or more cells forming spherical groups in the soft agar. The percentage efficiency was determined by the ratio of number of cell colonies counted divided by the total cell number plated.

TABLE 3
Colony formation in soft agar

| Cell line | % Efficiency |
|---|---|
| HEP 2 | 22.90 |
| VERO (passage 129) | 12.70 |
| MDCK (parental line) | 0.70 |
| MDCK.5F1 (passage 12) | 0.42 |
| HDCK.5F1 (passage 30) | 0.51 |
| MRC-5 (neg. control) | 0.00 |

The results of Table 3 show that the MDCK.5 μl cell line grows with minimal efficiency in soft agar, an indication that it may be non-tumorigenic in animals. This property was maintained 18 passages later, demonstrating that the phenotype is stable.

To further support this finding of non-tumorigenicity, we tested the tumor formation potential of MDCK.5F1 cell line in athymic nude mice.

### Example 5

### Evaluation of Tumor Formation in Nude (nu/nu) Athymic Mice following Subcutaneous Inoculation of Cell Suspension of MDCK.5F1 Clone

Nude (nu/nu) athymic mice fail to mount a cell mediated response against foreign materials and therefore will support the growth of allogeneic and heterogeneic tumor cell lines. This permits the assessment of the capability of an inoculum to form neoplasms in vivo.

Six week old female nude mice were inoculated subcutaneously with approximately 1×10⁷ cells of the test article, MDCK.5F1, followed clinically for 84 days and necropsied. Nude mice inoculated with positive control cells and negative control cells were treated similarly. The inoculation site (skin), lung, scapular lymph nodes and gross lesions were processed, sectioned, stained and examined microscopically. Further particulars of the experiment are presented below.

### Inoculation of Test and Control Materials

All mice within each cage were treated identically.

Each mouse was inoculated subcutaneously between the scapulae with 0.2 ml of the appropriate inoculum as described below. A 22 gauge needle was used for inoculation and all mice were inoculated on the same day.
Groups 1 and 2: Test article, MDCK.5F1 (at a concentration of 5×10⁷ cells/ml).
Groups 3 and 4: Positive control (18C1-10T cells at a concentration of 5×10⁷ cells/ml).
Groups 5 and 6: Negative control (SHE cells at a concentration of 1×10⁷ cells/ml).

All animals were observed every working day and the inoculation site palpated twice a week for a period of up to 84 days.

### Results

### Clinical Findings

All positive control mice were sacrificed and necropsied 14 days post inoculation, because all had large masses with at least one dimension greater than 1 cm at the inoculation site. All negative control mice were sacrificed and necropsied 84 days post-inoculation.

Nine out of 10 test article (5F1) mice were sacrificed and necropsied 84 post-inoculation. One of the 5F1 inoculated mice was sacrificed and necropsied 33 days post-inoculation, because the lesion at the inoculation site, which was progressing began to regress. This lesion was later revealed to be a cyst.

### Palpation

The ten nude mice inoculated with the positive control article had palpable lesions with at least one dimension greater than 1 cm by day 14 post-inoculation.

Small non-progressing lesions were palpable at the inoculation sites of the ten negative control article inoculated nude mice. These lesions first noted day 4 post-inoculation persisted in eight of the ten negative control mice for the duration of the observation period.

Palpation results are summarized in Table 4.

All of the ten 5F1 mice had lesions by day 4 post-inoculation. In nine of the ten 5F1 mice these lesions were small and did not progress. By day 56 post-inoculation, there were no palpable lesions in eight of the test article mice. One 5F1 inoculated mouse had an inoculation site lesion which progressed significantly in size between day 25 and day 28 and had markedly decreased in size by day 32 post-inoculation. This lesion was identified as a cyst by microscopic examination (see Table 6). The other mouse presenting a lesion had a localized inflammation.

TABLE 4
Palpation results

| Days Post-Inoculation | | | | | | | | | | | | |
|---|---|---|---|---|---|---|---|---|---|---|---|---|
| 4 | 7 | 11 | 14 | 18 | 21 | 25 | 28 | 32 | 35 | 39 | 41 | 46 |

5F1

| | | | | | | | | | | | | | |
|---|---|---|---|---|---|---|---|---|---|---|---|---|---|
| Lesion | 10/10 | 10/10 | 10/10 | 10/10 | 10/10 | 10/10 | 7/10 | 7/10 | 6/10 | 4/9 | 4/9 | 4/9 | 3/9 |
| Incidence | | | | | | | | | | | | | |
| Range of | 4-9 | 4-9 | 4-7 | 4-7 | 2-9 | 2-9 | 3-6 | 3-10 | 3-5 | 3-6 | 2-5 | 2-5 | 4-5 |
| Maximum | | | | | | | | | | | | | |
| Dimension of | | | | | | | | | | | | | |
| Lesions (mm) | | | | | | | | | | | | | |

Positive Control

| | | | | | | | | |
|---|---|---|---|---|---|---|---|---|
| Lesion | 10/10 | 10/10 | 10/10 | 10/10 | — | — | — | — |
| Incidence | | | | | | | | |
| Range of | 4-10 | 6-11 | 8-16 | 10-25 | — | — | — | — |
| Maximum | | | | | | | | |
| Dimension of | | | | | | | | |
| Lesions (mm) | | | | | | | | |

Negative Control

| | | | | | | | | | | | | | |
|---|---|---|---|---|---|---|---|---|---|---|---|---|---|
| Lesion | 10/10 | 10/10 | 10/10 | 9/10 | 8/10 | 10/10 | 6/10 | 7/10 | 10/10 | 9/10 | 9/10 | 9/10 | 7/10 |
| Incidence | | | | | | | | | | | | | |
| Range of | 4-10 | 3-9 | 3-6 | 2-6 | 2-7 | 1-7 | 1-6 | 2-6 | 2-6 | 2-6 | 2-6 | 2-5 | 2-5 |
| Maximum | | | | | | | | | | | | | |
| Dimension of | | | | | | | | | | | | | |
| Lesions (mm) | | | | | | | | | | | | | |

| | Days Post-Inoculation | | | | | | | | | | |
|---|---|---|---|---|---|---|---|---|---|---|---|
| | 49 | 53 | 56 | 60 | 63 | 68 | 70 | 74 | 78 | 81 | 84 |
| 5F1 | | | | | | | | | | | |
| Lesion | 5/9 | 2/9 | 1/9 | 1/9 | 2/9 | 2/9 | 1/9 | 1/9 | 1/9 | 2/9 | 3/9 |
| Incidence | | | | | | | | | | | |
| Range of | 3-4 | 3-4 | 4 | 3 | 3 | 3 | 2 | 2 | 3 | 3 | 2-3 |
| Maximum | | | | | | | | | | | |
| Dimension of | | | | | | | | | | | |
| Lesions (mm) | | | | | | | | | | | |
| Positive Control | | | | | | | | | | | |
| Lesion | | | | | | | | | | | |
| Incidence | | | | | | | | | | | |
| Range of | | | | | | | | | | | |
| Maximum | | | | | | | | | | | |
| Dimension of | | | | | | | | | | | |
| Lesions (mm) | | | | | | | | | | | |
| Negative Control | | | | | | | | | | | |
| Lesion | 6/10 | 7/10 | 7/10 | 8/10 | 6/10 | 7/10 | 9/10 | 9/10 | 8/10 | 7/10 | 8/10 |
| Incidence | | | | | | | | | | | |
| Range of | 4 | 3-5 | 2-5 | 3-5 | 3-5 | 2-4 | 1-5 | 1-5 | 2-5 | 2-5 | 2-5 |
| Maximum | | | | | | | | | | | |
| Dimension of | | | | | | | | | | | |
| Lesions (mm) | | | | | | | | | | | |

### Gross Necropsy Findings

Treatment-related gross necropsy findings are summarized in TABLE 5.

TABLE 5
Treatment related gross findings

| | | Positive | Negative |
|---|---|---|---|
| Organ/Lesion | 5F1 | Control | Control |
| Skin Inoculation | (10) | (10) | (10) |
| Site | | | |
| (No. Examined) | | | |
| Mass or Nodule | 3 | 10 | 6 |

Masses at the inoculation sites were found in all positive control animals. Masses or nodules were found at the inoculation sites in three of the ten 5F1 mice and six of the ten negative control mice.

### Microscopic Findings

Lesions of interest are summarized in TABLE 6.

TABLE 6
Treatment-related microscopic findings.

| | | Positive | Negative |
|---|---|---|---|
| Organ/Lesions | 5F1 | Control | Control |
| Skin Inoculation Site | (10) | (10) | (10) |
| (No. Examined) | | | |
| Fibrosarcoma | 0 | 10 | 0 |
| Osseous Proliferation | 0 | 0 | 2 |
| Inflammation, | 1 | 0 | 0 |
| subacute | | | |
| Cyst | 1 | 0 | 0 |
| No corollary change | 1 | 0 | 4 |
| detected | | | |

Neoplasia (fibrosarcoma) was diagnosed at the inoculation site in all positive control mice. The fibrosarcoma consisted of spindloid cells arranged in bundles of variable density with an interweaving pattern. Collagen deposition was minimal. Adjacent tissues were compressed, but seldom invaded by the neoplasm.

No neoplasms were diagnosed in any negative control mice, however, foci of osseous proliferation were noted at the inoculation sites in two of the negative control article mice. This is thought to represent select differentiation and growth of the SHE cells inoculated as the negative control.

No neoplasms were diagnosed in any 5F1 mice, however, a cyst was noted in one mouse and focal subacute inflammation was noted in one other 5F1 mouse.

### CONCLUSIONS

Fibrosarcoma was diagnosed at the inoculation site of all ten of the positive control mice.

No neoplasms were present in any of the negative control or test article mice.

Under the conditions of the above study, the test article, MDCK.5F1, is not considered to be tumorigenic.

### Example 6

### General Procedures

### a) Cell Multiplication

Before seeding the bioreactor, MDCK.5F1 (ATCC No. CRL-12042) cells were passaged a few times for cell amplification. Approximately 5-10×10⁶ cells were thawed in a 37° C. water bath, transferred to a polystyrene cell culture flask with nutritive medium and incubated at 37° C. After 3-4 days, cells from this flask were dissociated and used to seed 5 other flasks. The 5 flasks were then used to seed 20 flasks in the same manner. The nutritive medium used for cell growth was Dulbecco's Modified Eagle Medium and Medium 199 in a 1:1 ratio prepared in deionized water and containing 4.5 g/L of glucose, 0.58 g/L of glutamine and 1 g/L of sodium bicarbonate (DMEM-199). The nutritive medium was supplemented with 10% gamma-irradiated fetal bovine serum (1-FBS). The solution used to dissociate the cells in the flasks for cell passaging was a 0.25% trypsin with 0.02% ethylene diamine tetraacetic acid (EDTA) solution prepared in phosphate buffered saline (PBS) without magnesium and calcium.

The cells in the 20 flasks were grown for 3-4 days, trypsinized, collected and used to seed 3 microcarrier cell cultures in 1000-ml spinner flasks containing 3-5 g/L of microcarrier beads. The spinner flasks were incubated at 37° C. with stirring maintained at about 50 rpm. Cell growth was continued for 5-7 days after which the cells were trypsinized from the microcarriers and used to seed a 5-L bioreactor (CelliGen™ by New Brunswick of Edison, N.J.).

Trypsinization of the cells from the microcarriers was done in the following way. The microcarrier cell culture was washed twice with a solution of PBS and 0.02% EDTA. After the second cell wash, approximately 200 ml of trypsin solution was poured into the flask and left at 37° C. for about 20 minutes with stirring. After cell dissociation was complete, as determined by light microscopy, the cells were recovered from the free microcarriers using DMEM-199 containing 2% I-FBS, then pelleted and resuspended in DMEM-199 containing 10% I-FBS. A cell count was performed and an appropriate number of cells (approximately 10¹⁰) used to seed the bioreactor.

The spherical beads or microcarriers used for the cell cultures are manufactured by Pharmacia (Sweden) and distributed under the trade name of Cytodex 1. The density of the Cytodex 1 microcarriers was 1.03 (g/ml in 0.9% NaCl) and their size varied between 131 and 220 μm, with an average of 180 μm. The approximate surface area for cell growth was 4,500 cm²/g microcarrier (dry weight), with one gram containing approximately 6.8×10⁶ microcarriers.

### b) Cell Seeding and Growth in the Bioreactor

A concentration of 15-25 g/L of Cytodex 1 microcarriers was introduced in a 5-L bioreactor (3.7 L working volume). Seeding of the bioreactor was performed as follows. Approximately 4×10⁹−1×10¹⁰ cells obtained from the previously prepared stock (see above) were placed in a tubular glass bottle. From a sterile solution of 20 g/L of microcarriers, 55.5-92.5 g of microcarriers (depending on the desired concentration in the culture) were rinsed twice with DMEM-199 and added to the cells in the glass bottle. The bottle was subsequently filled with DMEM-199 containing 10% I-FBS to a final volume of 3.7 L. The contents of the bottle (cells, microcarriers in DMEM-199) were then poured into the bioreactor vessel with the central shaft rotating at about 20 rpm. With the vessel filled, stirring was increased to 50 rpm, the temperature adjusted to 37° C. and the dissolved oxygen content maintained between 5-50% of air saturation. The pH of the culture was also maintained at 6.8-7.4. Perfusion of the microcarrier cell culture was started on day 1 at 0.5 volumes/day using DMEM-199 with 2.5% I-FBS and 0.5 g/L magnesium sulfate. Cell growth was continued for approximately 7-10 days and the perfusion flow rate was gradually increased to 2 volumes/day.

### c) Virus Infection

Before the virus was added to the microcarrier cell culture, perfusion was increased to 4 serum-free volumes/day on the day of infection, the temperature lowered to 33° C. and the partial pressure of oxygen controlled at 15% air saturation. At cell confluency and right before virus infection (on the same day), the culture medium was replaced with the same culture medium free of serum and the perfusion rate increased to 4 volumes/day for 7 hours. This ensured that the serum content of the culture was reduced to a minimal level before infection. After that period, perfusion was stopped and human influenza virus of type A or B was introduced into the microcarrier cell culture. The virus was normally diluted with nutritive medium (DMEM-199 medium containing 6.5 g glucose/L) before introduction in the bioreactor in order to obtain a M.O.I. in the range of 1:10 to 1:10⁸. The next day, perfusion was maintained at 2 volumes/day until the cytopathic effect was complete. Total destruction of MDCK cells was typically observed within 5 days. The effluent containing influenza virus suspension was then collected and processed to produce a vaccine, as is well known in the art.

### d) Inactivation and Purification

Purification of monovalent influenza virus was performed in the following manner. The virus harvest collected from the bioreactor, generally between 15-30 L, was first clarified through a 1.2 μm filter (Sartorius Sartopure GF®, 10 inch in length, 0.6 m²) in order to remove large cellular debris. The clarified suspension containing the influenza virus was subsequently inactivated by the addition of 0.125% (V/V) formaldehyde (final concentration) for 16 hours. The inactivated viral suspension was then purified by ion exchange, DNAase treatment and gel filtration.

Enriched virus fractions were selected and represented the premium material to be used in vaccine preparation. These fractions were then pooled and diluted to give a final concentration of 15 μg of HA per strain per dose as currently recommended by international authorities.

### e) Vaccine Preparation

Viral proteins were diluted to 15 μg for each dose of vaccine. Thimerosal (0.01%) was added for preservation and stabilization, respectively, to complete the vaccine.

For a standard trivalent vaccine, a monovalent dose of each of the three circulating strains would be mixed and added to the preservative agent and stabilizer described above.

Other known preservative agents such as aminomethyl propanol, sorbic acid and polyaminopropyl biguanid, phenymercuric nitrate, phenylmercuric borate, 2-phenoxyethanol with formaldehyde, phenol, benzethonium chloride and 2-phenoxyethanol can be used for the vaccine preparation. Concentration of these preservative agents will need to be acceptable to industry standards.

### Example 7

### Infection of Parental Cell Line with Strain A/Shanghai/11/87 with the Addition of Trypsin

MDCK cells from the parental line were grown in a CelliGen™ bioreactor. The working volume of the bioreactor was 3.7 L, the microcarrier concentration was 25 g/L and stirring was set at 50 rpm. On the seventh day, the culture was infected with human influenza virus designated A/Shanghai/11/87. The M.O.I. was 1:133,000 and 2.5 μg/ml trypsin was added to enhance viral replication. TABLE 7 sets out the data relating to the assay while TABLE 8 summarizes the results. The yield of vaccine was 9,828 monovalent doses of 15 μg HA, based on a total harvest volume of 18 L and Single Radial Diffusion (SRD) assay values of 7.38 μg HA/ml and 9 μg HA/ml. (See TABLE 8.)

TABLE 7
Data for assay with parental cell line and strain
A/Shanghai/11/87 with the addition of trypsin

| | | | | | | | | | Cell |
|---|---|---|---|---|---|---|---|---|---|
| | | | | | | | | | Concn |
| | Post- | | | Dissolved O₂ | Air | O₂ | | | (x |
| Culture | Infection | Temp | | (% air | Pressure | Pressure | Perfusion | % | 10⁶ cells/ |
| (days) | (days) | (° C.) | pH | saturation) | (psi) | (psi) | (vol/day) | Serum | ml) |
| 0 | | 37 | 6.40 | 5 | <<1 | <<1 | | 10 | 1.45 |
| 0.16 | | 37 | 6.40 | 5 | <<1 | <<1 | 1 | 5 | |
| 1 | | 37 | 6.53 | 5 | <<1 | <<1 | 2 | 5 | 1.38 |
| 2 | | 37 | 6.90 | 5 | <<1 | <<1 | 2 | 5 | 1.67 |
| 3 | | 37 | 7.01 | 5 | <<1 | <<1 | 3 | 5 | |
| 4 | | 37 | 6.95 | 2 | <<1 | <<1 | 3 | 5 | |
| 5 | | 37 | 6.96 | 3 | <<1 | 2.5 | 3 | 5 | 6.26 |
| 6 | | 37 | 6.70 | 3 | <<1 | 2.5 | 3 | 0 | 7.69 |
| 7 | 0 | 34 | 6.50 | 5 | <<1 | 1.5 | 3 | 0 | 9.34 |
| 8 | 1 | 34 | 6.80 | 5 | <<1 | 1.5 | 2 | 0 | |
| 9 | 2 | 34 | 6.70 | 5 | <<1 | 1.5 | 2 | 0 | |
| 10 | 3 | 34 | 7.20 | 5 | <<1 | 1.5 | 2 | 0 | |
| 11 | 4 | 25 | 7.24 | 68 | <<1 | 1.51 | | | |
| 12 | 5 | 25 | 7.40 | 57 | <<1 | 1.5 | | | |

The total number of cells at the moment of infection is calculated in the following way: 9.34×10⁶ MDCK cells/ml×3700 ml equals 34,558×10⁶ cells.

TABLE 8
Results of assay with parental cell line and strain
A/Shanghai/11/87 with the addition of trypsin

| | | | | |
|---|---|---|---|---|
| | TCID₅₀/ml | SRD(μg | Volume | No. of |
| HA | (10^{x}) | HA/ml) | (L) | Doses |

| | | |
|---|---|---|
| Seed | 1.92 | 3.2 |

Bioreactor sample (time post-infection)

| | | | | |
|---|---|---|---|---|
| 17 | hrs. | neg. | ≦2.3 | |
| 2 | days | 192 | 3.8 | |
| 3 | days | 512 | 3.8 | |
| 5 | days | 192 | 3.2 | 13.2 |

Harvest sample (time post-infection)

| | | | | | | |
|---|---|---|---|---|---|---|
| 0-24 | hrs. | neg. | ≦1.8 | | | |
| 1-2 | days | 64 | 4.0 | neg. | | |
| 2-3 | days | 512 | 3.9 | 7.38 | 9 | 4,428 |
| 3-4 | days | 384 | 3.9 | 9.00 | 9 | 5,400 |

| | | |
|---|---|---|
| Total | 18 | 9,828 |

Calculation of the total amount of HA is determined in the following way: 9,828 doses×15 μg/dose=147,420 μg HA total. When divided by 34558×10⁶ cells=4.26 μg HA/10⁶ MDCK cells.

### Example 8

### Infection of MDCK.5F1 Clone with Strain A/Shanghai/11/87 without Trypsin

Cells derived from clone MDCK.5F1 (ATCC number CRL-12042) were grown in a CelliGen™ bioreactor with a microcarrier concentration of 25 g/L. The working volume of the bioreactor was 3.7 L and stirring was set to 50-55 rpm. On the seventh day, the culture was infected with human influenza virus designated A/Shanghai/11/87. No trypsin was added to the bioreactor to enhance the growth of the virus. The M.O.I. was 1:133,000 and the assay produced 32 L with an SRD value of 9.2 μg HA/ml, resulting in 19,626 monovalent doses. TABLE 9 summarizes the data for the assay and TABLE 10 lists the results.

TABLE 9
Data for assay with MDCK.5F1 clone and strain
A/Shanghai/11/87 in the absence of trypsin

| | | | | | | | | | Cell |
|---|---|---|---|---|---|---|---|---|---|
| | | | | | | | | | Concn |
| | Post- | | | Dissolved O₂ | Air | O₂ | | | (x |
| Culture | Infection | Temp | | (% air | Pressure | Pressure | Perfusion | % | 10⁶ cells/ |
| (days) | (days) | (° C.) | pH | saturation) | (psi) | (psi) | (vol/day) | Serum | ml) |
| 0 | | 37 | | 5 | <1 | <1 | | 10 | 2.3 |
| 1 | | 37 | 6.88 | 5 | <1 | <1 | 2 | 5 | 1.9 |
| 2 | | 37 | 6.94 | 5 | <1 | 1 | 2 | 5 | |
| 3 | | 37 | 6.59 | 5 | <1 | 1 | 3 | 5 | |
| 4 | | 37 | 6.85 | 5 | <<1 | 1 | 3 | 5 | 7.9 |
| 5 | | 37 | 6.84 | 4 | <<1 | 3 | 3 | 5 | 11 |
| 6 | | 37 | 6.82 | 4 | <<1 | 3 | 3 | 5 | 14.4 |
| 7 | 0 | 33 | 6.83 | 4 | <<1 | 3.5 | 3-4 | 0 | 16.8 |
| 8 | 1 | 33 | 6.93 | 4 | <<1 | 3.5 | 2 | 0 | |
| 9 | 2 | 33 | 6.95 | 7 | <<1 | 3.5 | 2 | 0 | |
| 10 | 3 | 33 | 7.23 | 7 | <<1 | 3.5 | 2 | 0 | |
| 11 | 4 | 33 | 7.31 | 6-7 | <<1 | 3.5 | 2 | 0 | |

The total number of cells at the moment of infection is calculated in the following way: 16.8×10⁶ MDCK cells/ml×3700 ml equals 62,160×10⁶ cells.

TABLE 10
Results of assay with MDCK.5F1 clone and strain
A/Shanghai/11/87 in the absence of trypsin

| | | | | |
|---|---|---|---|---|
| | TCID₅₀/ml | SRD(μg | Volume | No. of |
| HA | (10^{x}) | HA/ml) | (L) | Doses |

| | | |
|---|---|---|
| Seed | 512 | 14.21 |

Bioreactor sample (time post-infection)

| | | | | |
|---|---|---|---|---|
| 17 | hrs. | neg. | 3.4 | |
| 2 | days | 384 | 6.1 | 7.63 |
| 3 | days | 512 | 5.1 | 13.94 |
| 4 | days | 128 | 5.2 | 6.87 |

Harvest sample (time post-infection)

| | | | | | | |
|---|---|---|---|---|---|---|
| 0-4 | days | 192 | 5.2 | 9.2 | 32 | 19,626 |

| | | |
|---|---|---|
| Total | 32 | 19,626 |

Calculation of the total amount of HA is determined in the following way: 19,626 doses×15 μg/dose=294,390 μg HA total. When divided by 62,160×10⁶ cells=4.73 μg HA/10⁶ MDCK cells.

### Example 9

### Infection of MDCK.5F1 Clone with Strain B/Harbin/7/94 without Trypsin

Cells derived from clone MDCK.5F1 were grown as described in EXAMPLE 3 but with a microcarrier concentration of 15 g/L. On the eighth day, the culture was infected with human influenza virus designated B-Harbin/7/94. The M.O.I. was 1:10,000. No trypsin was added to promote viral replication. Data and results from this assay are reproduced in TABLES 11 and 12. The yield was 38,150 doses, based on a harvest of 35 L and an SRD value of 16.35 μg HA/ml.

TABLE 11
Data for assay with MDCK.5F1 clone and strain B-
Harbin/7/94 without trypsin

| | | | | | | | | | Cell |
|---|---|---|---|---|---|---|---|---|---|
| | | | | | | | | | Concn |
| | Post- | | | Dissolved O₂ | Air | O₂ | | | (x |
| Culture | Infection | Temp | | (% air | Pressure | Pressure | Perfusion | % | 10⁶ cells/ |
| (days) | (days) | (° C.) | pH | saturation) | (psi) | (psi) | (vol/day) | Serum | ml) |
| 0 | | 37 | | 8 | <1 | <1 | | 10 | 1.20 |
| 1 | | 37 | 6.87 | 5 | <1 | <1 | 0.5 | 2.5 | 1.79 |
| 2 | | 37 | <7.0 | 5 | | | 0.5 | 2.5 | 2.66 |
| 3 | | 37 | ≈7.0 | 5 | | | 0.5 | 2.5 | |
| 4 | | 37 | ≈7.0 | 4 | | | 1 | 2.5 | |
| 5 | | 37 | ≈7.0 | 5 | | | 1.5 | 2.5 | 3.88 |
| 6 | | 37 | ≈7.0 | 3 | | | 1.5 | 2.5 | 11.37 |
| 7 | | 37 | ≈7.0 | 2.3 | | | 2 | 2.5 | 13.33 |
| 8 | 0 | 33 | ≈7.1 | 2 | | | 4 | 0 | 16.70 |
| | | | | 13 | | | 3 | | |
| 9 | 1 | 33 | 7.15 | 15 | | | 2 | 0 | |
| 10 | 2 | 33 | 7.1 | 16 | | | 2 | 0 | |
| 11 | 3 | 33 | 7.2 | 16 | | | 2 | 0 | |
| 12 | 4 | 33 | ≧7.2 | | | 1.5 | 2 | 0 | |
| | | | | | | | 4 | | |

The total number of cells at the moment of infection is calculated in the following way: 16.7×10⁶ MDCK cells/ml×3700 ml equals 61,790×10⁶ cells.

TABLE 12
Results of assay with MDCK.5F1 clone and strain B-
Harbin/7/94 in the absence of trypsin

| | | | | |
|---|---|---|---|---|
| | TCID₅₀/ml | SRD(μg | Volume | No. of |
| HA | (10^{x}) | HA/ml) | (L) | Doses |

| | | |
|---|---|---|
| Seed | 128 | 7.22 |

Bioreactor sample (time post-infection)

| | | | | |
|---|---|---|---|---|
| 19 | hrs. | neg. | 7.3 | |
| 2 | days | 2048 | 8.72 | |
| 3 | days | 1024 | 7.88 | |
| 4 | days | 96 | 5.97 | <7.885 |

Harvest sample (time post-infection)

| | | | | | | |
|---|---|---|---|---|---|---|
| 0-4 | days | 512 | 7.88 | 16.35 | 35 | 38,150 |

| | | |
|---|---|---|
| Total | 35 | 38,150 |

Calculation of the total amount of HA is determined in the following way: 38,150 doses×15 μg/dose=572,250 μg HA total. When divided by 61,790×10⁶ cells=9.26 μg HA/10⁶ MDCK cells.

Once again, it is shown that the process of this invention gives yields that are equal to or greater than yields given by prior art processes that require the presence of trypsin.

1. A Madin-Darby Canine Kidney (MDCK)—derived cell line characterized in that it has a higher susceptibility to viral infection than its parental MDCK cell line.

2. The cell line according to claim 1, wherein said higher susceptibility is defined as at least about 1.2 times the titer of virus produced in said parental cell line.

3. The cell line according to claim 2, wherein said virus is selected from the group consisting of: influenza, respiratory syncytial virus, papovavirus, parainfluenza, vesicular stomatitis, vaccinia, Coxsackie, reovirus, parvovirus, adenovirus, poliomyelitis, measles, rabies and herpes viruses.

4. The cell line according to claim 3, wherein said virus is influenza or respiratory syncytial virus.

5. The cell line according to claim 4, wherein said influenza virus comprises human, equine, porcine, or avian strains.

6. The cell line according to claim 5, wherein said influenza virus is selected from human influenza virus types A, B, or C.

7. The cell line according to claim 6, wherein said influenza virus is selected from type A or B.

8. The cell line according to claim 7, wherein said cell line is highly susceptible to both influenza virus types A and B.

9. A MDCK-derived cell line characterized in that it is non-tumorigenic.

10. The cell line according to any one of claims 1 to 8, characterized in that it is non-tumorigenic.

11. The cell line according to claim 10, wherein said non-tumorigenicity is defined as an absence of palpable nodules in nude mice after about 3 months of observation.

12. The cell line which has the biological properties of ATCC CRL-12042.

13. The cell line defined as ATCC CRL-12042.

14. The cell line according to any one of claims 1 to 13, further characterized in that it is anchorage-dependent.

15.-23. (canceled)

24. A process for the production of influenza virus particles or proteins, comprising;
a) growing a Madin-Darby Canine Kidney (MDCK) cell line capable of replicating said influenza virus;
b) infecting said cell line with a strain of influenza virus and
c) incubating to allow replication of said virus; and
d) harvesting said replicated virus.

25. The process of claim 24 which further comprises purifying virus particles or proteins there from.

26. The process of claim 24, wherein said MDCK cell line is defined as having the biological properties of ATCC No. CRL-12042.

27. The process of claim 24, wherein said MDCK cell line is defined as ATCC No. CRL-12042.

28. The process of claim 24, wherein said influenza virus comprises human, equine, porcine or avian strains.

29. The process of claim 24, wherein said virus is selected from the group consisting of human influenza types A, B and C.

30. The process of claim 24, wherein said process is carried out in a bioreactor.

31. The process of claim 30, wherein said bioreactor is about 5 liters in size.

32. The process of claim 30, wherein said bioreactor is from about 5 to 5000 liters in size.

33. The process of claim 24, wherein in step a), said cells are grown in medium containing microcarrier means, and in step b), said incubation is carried out with a perfusing means.

34. The process of claim 33, wherein said perfusing means comprises a decanter to avoid leakage of microcarriers in said harvest.

35. The process of claim 33, wherein said microcarrier means comprises microcarrier beads at a concentration of about 5-25 g/L.

36. The process of claim 35, wherein said microcarrier bead concentration is in the range of about 10-25 g/L.

37. The process of claim 35, wherein said microcarrier bead concentration is in the range of about 15-20 g/L.

38. The process of claim 34, wherein said perfusing is carried out at a rate of about 0.5-4.0 volumes of bioreactor per day.

39. The process of claim 24, wherein in step b), said influenza virus is seeded at a multiplicity of infection of about 10:1-1:10¹⁰.

40. The process of claim 24, wherein said influenza virus is seeded at a multiplicity of infection of about 1:10-1:10⁸.

41. The process of claim 24, wherein said influenza virus is seeded at a multiplicity of infection of about 1:10⁴-1:10⁷.

42. The process of claim 24, wherein said influenza virus is seeded at a multiplicity of infection of about 1:10⁵-1:10⁶.

43. The process of claim 24, wherein in step a), said MDCK cell line is grown for about 7 days.

44. The process of claim 43, wherein said MDCK cell line is grown at a temperature in the range of about 33-40° C.

45. The process of claim 43, wherein said MDCK cell line is grown at a temperature in the range of about 36-38° C.

46. The process of claim 24, wherein in step b), said influenza virus is replicated at a temperature of about 30-37° C.

47. The process of claim 43, wherein said influenza virus is replicated at a temperature of about 32-34° C.

48. The process of claim 43, wherein said influenza virus is replicated at a temperature of about 33° C.

49. The process of claim 24, wherein said purified virus particles or proteins are used to produce a vaccine to prevent influenza virus infections.

50. A vaccine for the prevention of influenza virus infection in a mammal, as made by the process of claim 49.

51. The vaccine of claim 50, wherein said mammal is a human.

52. A method for the prevention of influenza infection in a mammal, comprising the step of administering the vaccine of claim 50 or 51.

53. The vaccine of claim 50, wherein the vaccine comprises the virus particles or virus proteins in admixture with a preservative agent.

54. (canceled)

55. The process of claim 24, wherein said purified viral particles or proteins are used to produce a diagnostic kit for the detection of influenza virus infections.

56. (canceled)
